(19) United States
(12) Patent Application Publication
(10) Pub. No.: US 2008/0243167 A1
(43) Pub. Date: Oct. 2, 2008

(54) Intragastric Balloon With Improved Forming Means and Increased Mechanical Strength

(75) Inventors: Pascal Paganon, Serezin du Rhone (FR); Jean-Paul Gilbert Ricol, Saint Germain sur l'Arbresle (FR)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: Compagnie Europeenne d'Etude et de Recherche de Dispositifs pour I'Implantation par Pararoscopie, Vienne (FR)

(21) Appl. No.: 10/598,103

(22) PCT Filed: Feb. 18, 2005
(86) PCT No.: PCT/FR2005/000389

§ 371 Date: Apr. 7, 2008

(30) Foreign Application Priority Data
Feb. 20, 2004 (FR) 0401733

(51) Int. Cl. A61M 29/02

(52) U.S. Cl. 606/192

(57) ABSTRACT

An expandable intragastric balloon implantable in the stomach for the treatment of obesity, comprising an outer casing (2) which is sufficiently flexible to change from a reduced volume configuration to an expanded configuration, thereby imparting the balloon (1) with its functional shape. The balloon further comprises means (3) for forming the outer casing (2), which are structurally integrated into the balloon (1), but generally are separate from the outer casing (2). Once the balloon (1) has been implanted, the aforementioned forming means (3) can be actuated in order to: (i) exert a sufficient driving pressure on the outer casing (2) such as to force the deployment thereof, and (ii) occupy a sufficient volume inside the outer casing (2) in order to produce the deployment of the outer casing (2) from the reduced volume configuration to the expanded configuration thereof.

### TECHNICAL FIELD

This invention relates to the general technical field of devices that can be implanted in the human body and that are designed to be used for the treatment of obesity, and particularly morbid obesity, and most particularly to implants capable of artificially reducing the volume of the stomach for the particular purpose of producing a sensation of fullness in the patient.

This invention relates to an expandable intragastric balloon designed to be implanted in the stomach of a patient for the treatment of obesity, and comprising an outer casing that is sufficiently flexible to pass from a reduced-volume configuration to an expanded configuration, thereby imparting the balloon with its functional shape.

### PRIOR ART

Intragastric balloons used for the treatment of obesity are generally in the form of a flexible pouch forming the outer casing of the balloon and capable of being filled with an inflating fluid, in particular air, once the balloon has been implanted inside the stomach, in order to impart the balloon with its functional shape, i.e., an operating volume and shape that enable it to occupy a large portion of the space available for food.

Although they have many advantages, linked in particular to their ease of manufacture, such balloons nevertheless suffer from several disadvantages.

In particular, known intragastric balloons often have a certain degree of porosity, which will enable gradual leaking of the fluid contained inside the balloon (gas or liquid), and bring about the gradual reduction in the volume of the balloon, and thus the therapeutic effectiveness thereof.

Thus, when the volume of the balloon no longer corresponds to the volume desired for the treatment of obesity, the latter no longer creates the desired sensation of fullness in the patient.

This problem proves to be particularly critical in the case where the outer casing of the balloon is filled only with gas.

In certain extreme cases, the reduction in the volume of the balloon may cause the latter to pass through the pylorus and into the intestinal tract, which causes severe complications in the patient.

The properties or characteristics of the intragastric balloon, such as its leak tightness, which are responsible for maintaining the balloon in it functional configuration over time, thus appear to be essential parameters the control of which makes it possible to improve the effectiveness of the medical treatment.

Therefore, there is an entirely obvious interest in producing a balloon designed to preserve its volume and functional configuration over time, and even when the outer casing, which is subjected to external stresses and, in particular, digestive juices, deteriorates and gradually becomes porous.

Furthermore, the intragastric balloons generally encountered likewise suffer from a certain degree of fragility, which, in particular, comes from the fact that the pouch containing the inflating gas (or the liquid) also forms the outer casing of the balloon. Thus, said pouch is subject directly to external stresses, which increases the risk of perforation, particularly when the balloon is being handled by the medical practitioner.

Therefore, there is an entirely obvious interest in producing an intragastric balloon that, while being sufficiently elastic, is likewise sufficiently mechanically strong and protected from external stresses.

### DISCLOSURE OF THE INVENTION

Consequently, the objects assigned to the invention aim to remedy the various disadvantages listed above and to propose a new, expandable intragastric balloon for the treatment of obesity, which does not have the above-listed disadvantages and which, once positioned inside the stomach in its functional configuration, has very slight variations in volume over the course of time.

Another object of the invention aims to propose a new intragastric balloon whose leak-tightness is improved, and whose losses of inflating fluid, in particular gas, are limited, thereby increasing the therapeutically effective lifespan of the balloon.

Another object of the invention aims to propose a new intragastric balloon that, while having good elastic properties, is sufficiently mechanically strong and protected from external stresses.

Another object of the invention aims to propose a new intragastric balloon whose forming and expansion are particularly simplified and fast.

Another object of the invention aims to propose a new intragastric balloon that, although having a considerable volume, is sufficiently lightweight, atraumatic and well-tolerated by the patient.

The objects assigned to the invention are achieved with the aid of an expandable intragastric balloon designed to be implanted in the stomach of a patient for the treatment of obesity and comprising an outer casing that is sufficiently flexible to pass from a reduced-volume configuration to an expanded configuration, thereby imparting the balloon with its functional shape, characterized in that it comprises means for forming the outer casing, which are structurally integrated into the balloon and primarily separate from the outer casing, said forming means being capable of being actuated, once the balloon has been implanted, on the one hand, in order to exert a sufficient driving pressure on the outer casing to force it to deploy and, on the other hand, to occupy a sufficient volume inside said outer casing to ensure the deployment of the outer casing from its reduced-volume configuration to its expanded configuration.

### SUMMARY DESCRIPTION OF THE DRAWINGS

Other objects and advantages of the invention will become more apparent upon reading the following description, as well as with the aid of the appended drawings, given for purely illustrative and non-limiting purposes, in which:

FIG. 1 is a cross-sectional view showing an intragastric balloon in accordance with the invention, in its maximum expansion position.

FIG. 2 is a cross-sectional view showing an intragastric balloon in accordance with the invention, in its unexpanded position.

### BEST MODE OF CARRYING OUT THE INVENTION

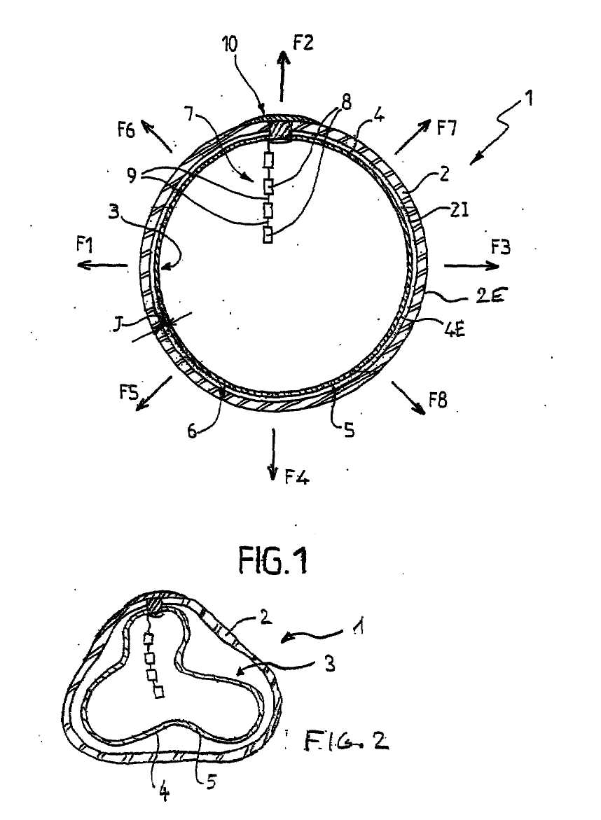

The intragastric balloon in accordance with the invention will now be described while referring to FIGS. 1 and 2.

The intragastric balloon 1 in accordance with the invention is designed to be implanted in the stomach of a patient for the treatment of obesity.

The intragastric balloon 1 is expandable and, for this purpose, comprises an outer casing 2 that is sufficiently flexible to pass from a reduced-volume configuration (shown in FIG. 2) to an expanded configuration (shown in FIG. 1).

The reduced-volume configuration, for example, may correspond to a configuration in which the balloon 1 is in a folded-over position occupying a reduced volume, thereby facilitating insertion of the balloon 1 into the esophagus.

As a matter of fact, implantation of the intragastric balloon 1 in accordance with the invention is generally performed, in a conventional manner well-known to those skilled in the art, by passing through the oral-esophageal route in its folded-over, compressed or loose form. The expansion, placement and release of the balloon occur at the end of the gastro-endoscopic operation, when the balloon 1 is correctly positioned inside the stomach of the patient.

Thus, it is in its expanded configuration that the balloon 1 will be able to occupy a considerable volume inside the gastric cavity (not shown), the so-called expanded configuration then imparting the balloon with its functional shape.

Thus, the expression “functional shape” refers, on the one hand, to the volume and, on the other hand, to the therapeutic operating shape of the balloon for the treatment of obesity.

Thus, by occupying a portion of the internal volume of the stomach, the intragastric balloon according to the invention makes it possible to create a rapid sensation of fullness in the patient, which is accompanied by a reduction in the amount of food ingested.

According to the invention, the intragastric balloon 1 comprises means for forming 3 the outer casing 2.

These forming means 3 are structurally integrated into the balloon, i.e., they are part of the balloon even before the latter is implanted inside of the gastric cavity. More precisely, the forming means 3 are integrated into the balloon 1 during its manufacture, thereby forming a portion of the functional components of the balloon.

According to the invention, once the balloon 1 has been implanted or put in place inside the stomach, the means for forming 3 the outer casing 2 are capable of being actuated:
on the one hand, in order to exert a sufficient driving pressure on the outer casing 2 to force it to deploy,
and, on the other hand, in order to occupy a sufficient volume inside said outer casing 2 to ensure deployment of the outer casing 2 from its reduced-volume configuration to its expanded configuration.

Thus, the forming means 3 are advantageously dimensioned so as to exert stress on the outer casing 2 with an intensity substantially greater than the resisting force capable of being opposed by said outer casing 2.

Within the meaning of the invention, the outer casing 2 designates the outside wall of the intragastric balloon 1, defined internally by an internal surface 2I and externally by an external surface 2E forming the contact surface of the balloon 1 with the stomach.

In a particularly advantageous way, the forming means 3 are configured and designed to exert a substantially normal and centrifugal stress on the outer casing 2, so as to push the latter in the direction of the arrows F1 to F6 shown in FIG. 1. In a particularly advantageous way, the stress is exerted over substantially the entire internal surface 2I of the outer casing 2, i.e., substantially in every direction in space.

The forming means 3 are advantageously separate from the outer casing 2, and designed to occupy an increasing volume inside the outer casing 2, so as to produce the deployment thereof. More precisely, the forming means 3 are primarily separate from the outer casing 2, with the result being that, when the balloon 1 is in an unexpanded or folded configuration, shown in FIG. 2, the forming means 3 are not necessarily in contact with the outer casing 2 over the entire internal surface 2I thereof.

The forming means 3 are thus advantageously mounted inside of the outer casing 2 with a possibility of being able to move in relation thereto.

The forming means 3 are thus advantageously designed to occupy a sufficient volume inside the outer casing 2, with the result being that the latter is able to attain its expanded configuration.

Preferentially, the forming means 3 have properties of flexibility and elasticity, which are particularly valuable in this field of application.

The design of the balloon 1 in accordance with the invention, and particularly the use of the forming means 3 described above, thus makes it possible to obtain an intragastric balloon 1 capable of retaining its functional shape over the course of time, even when the outer casing 2 gradually deteriorates.

According to a first possible embodiment of the invention, not shown in the figures, the forming means may advantageously consist of a viscoelastic memory foam having sufficient elastic recall properties in a centrifugal direction to deploy the outer casing of the balloon, when the latter is in an initial, reduced-volume configuration inside the stomach.

In this first embodiment, fluidic communication may be established, via a catheter, for example, between the inside of the outer casing and the atmosphere, so that the foam is able to expand while at the same time forcing the outer casing to deploy, air then being gradually drawn into the casing due to the expansion of the foam.

According to one preferential embodiment of the invention, shown in FIG. 1, the forming means 3 consist of an inflation chamber 4, which is different from the outer casing 2, and which is arranged inside thereof so as to ensure the formation of said outer casing 2 by the introduction of an inflating fluid into said inflation chamber 4.

The expression “inflation chamber” here designates an inflatable unit that is mounted inside the outer casing 2 and that defines an interior volume capable of being filled with an inflating fluid.

Preferably, the inflation chamber 4 and the outer casing 2 are primarily separate, and are advantageously connected or made integral with each other via a valve-type connection means 10 enabling connection of the inflation chamber 4 to a fluid reservoir, in order to fill the inflation chamber 4.

The inflation chamber 4 is thus capable of being actuated by inflation, with the aid of a fluid such as a gas (or else a liquid). As it inflates, the inflation chamber 4 will thereby expand and exert stress on the outer casing 2, specifically on the internal surface 2I thereof, tending to push it in a centrifugal direction, as shown by the arrows F1 to F6 in FIG. 1, and until the outer casing 2 attains its expanded and functional configuration.

The inflation chamber 4 and the outer casing 2 are thus shaped in such a way that, during the inflating operation, the outer casing 2 exerts only a slight resistance with regard to the pressure exerted on its internal surface 2I by the inflation chamber 4.

In this way, the inflation chamber 4 constitutes an impervious barrier between the inflating fluid that it contains and the outer casing 2, thereby preventing the inflating fluid from migrating towards the outer casing 2 of the balloon 1, whose leak tightness is weaker than the inflation chamber 4.

Furthermore, the outer casing 2 being separate and independent from the forming means 3, of the inflation chamber 4 specifically, it is capable of effectively ensuring the mechanical protection thereof.

For this purpose, the outer casing 2 will preferably be made of flexible, elastic materials having good mechanical strength properties.

In a particularly advantageous way, the inflation chamber 4 and the outer casing 2 are shaped such that, when the inflation chamber 4 occupies its expanded position, the outer casing 2 substantially matches the shape of said inflation chamber 4.

In this so-called expanded position, the inflation chamber 4 may be over-inflated, i.e., filled with an amount of fluid substantially greater than the amount of fluid that it is normally capable of containing without undergoing deformation or else elastic extension of its wall.

Preferentially, the inflation chamber 4 and the outer casing 2 are shaped such that, during the inflating operation, a slight clearance remains between the external surface 4E of the inflation chamber and the internal surface 2I of the outer casing 2, this clearance J being sufficient to allow a certain relative mobility between the inflation chamber 4 and the outer casing 2.

Such mobility thereby makes it possible, on the one hand, to prevent the outer casing 2 from interfering with the inflation of the inflation chamber 4 and, on the other hand, to prevent the formation of folds on the outer casing 2.

Advantageously, the inflation chamber 4 consists of an inner pouch 5 that is sufficiently flexible to pass from a reduced-volume position to an expanded position.

The inner pouch 5 preferentially consists of an elastomer material having a sufficient degree of elasticity to allow over-inflating of said inner pouch 5.

Advantageously, the inflation chamber 4, specifically the inner pouch 5, is designed to be filled with a gas forming the inflating fluid. Quite obviously, the inflating fluid might be a liquid, and without thereby exceeding the scope of the invention.

Thus, together with the inflation chamber 4 (or inner pouch 5) and the outer casing 2 constituting an outer pouch, the intragastric balloon 1 forms a double-pouch balloon.

Advantageously, the inner pouch 5 is defined by a wall 6, distinct and primarily separate from the outer casing 2, comprising at least one substantially gas-tight shield, so as to improve the overall leak tightness of the balloon. Thus, the wall 6 may advantageously consist of a multi-layer film, the shield then forming one of the film layers, or a single-layer film, in which case the shield forms the single layer of film.

In a particularly advantageous way, the shield includes in its composition at least one polymer having a gas barrier effect.

The expression “polymer having a gas barrier effect” designates polymers having a gas permeability clearly lower than that of silicone, and specifically an oxygen (O₂) permeability lower than approximately 10 times that of silicone.

Thus, the shield may consist of one or more thermoplastic polymers having a gas barrier effect, such as ethylene vinyl alcohol (EVOH), poly(vinylidene chloride) (PVDC), polyacrylonitrile (PAN), polyamide (PA), bi-oriented polyamide, poly(ethylene terephthalate) (PET), bi-oriented poly(ethylene terephthalate), and thermoplastic elastomer polyurethane.

According to one preferential alternative of the invention, the inner pouch 5 consists of one or more films, e.g., two thermoplastic elastomer polyurethane films, said films being capable of being made integral with each other, e.g., by gluing, or movable in relation to each other.

Among other valuable properties, polyurethane has a slight adhesive effect, which facilitates folding, and also shape memory properties.

As an illustrative example, the oxygen (O₂) permeability of polyurethane is of the order of 350 cm³·60 μm/m²·24 h·bar to 1,600 cm³·100 μm/m²·24 h·1 bar, while the permeability of silicone is of the order of 130,000 cm³·100 μm/m²·24 h·bar (measurements taken at 37° C. and 90% relative humidity).

A material such as this also has elastic properties allowing over-inflation of the inner pouch 5, while at the same time offering satisfactory mechanical strength.

Advantageously, the inflation chamber 4 is designed to have a substantially spherical shape in its expanded position.

For this purpose, the inner pouch 5 is preferentially manufactured using a heat-sealing, heat-welding or else heat-forming process.

More precisely, the method of manufacturing the inner pouch comprises an assembly stage, during the course of which one or more sheets (e.g., two sheets) are assembled by welding or gluing along a peripheral weld line, said sheets being of a predetermined shape and made of a material substantially impervious to gases, such as thermoplastic elastomer polyurethane that has been pre-formed, e.g., by heat-forming, so as to impart a hemispherical shape thereto.

Each sheet may consist of a single polyurethane film, but preferably consist of several, and, for example, two superimposed polyurethane films, said films being capable of being made integral with each other, e.g., by gluing, or movable in relation to each other.

As concerns the outer casing 2, it is preferentially made by means of an injection-molding process.

In a particularly advantageous way, the inflation chamber 4 and the outer casing 2 are substantially concentric and spherical, the spherical shape of the outer casing 2 thereby imparting an atraumatic property to the intragastric balloon 1.

According to one particularly advantageous characteristic of the invention, the outer casing consists of a bio-compatible material capable of being well-tolerated by the patient.

Thus, the outer casing 2 may consist of a silicone-type elastomer material, a material such as this having, in particular, valuable elastic and mechanical strength properties.

The outer casing 2 advantageously has radiological opacity properties.

Furthermore, according to one preferential alternative of the invention, the silicone used to form the outer casing 2 is advantageously colored white by treating with barium sulfate.

Preferentially, the thickness of the inner pouch 5 will be less than the thickness of the outer casing 2, the latter then providing a mechanical protection function with regard to the inner pouch 5, thereby limiting the risks of deterioration and/or perforation of said inner pouch 5.

Thus, the design of the balloon 1 according to the invention makes it possible to use two different materials to produce the inner pouch 5 and the outer casing 2, and to also produce an inner pouch 5 with a particularly small thickness, which makes it possible, in particular, to limit the space occupied by the balloon 1 when it is in its reduced-volume configuration for being implanted in the stomach of a patient.

In a particularly advantageous way, and in order to further improve the overall leak tightness of the balloon, it is possible to cover the outer casing 2 with parylene. Similarly, the inflation chamber 4 can also be covered with parylene.

A measure such as this also has the advantage of reducing the adhesive effect of the silicone.

According to a yet more preferential alternative of the invention, the intragastric balloon 1 comprises ballasting means consisting of solid and dense bodies 8, preferably made of tungsten, designed to substantially weigh down the balloon 1.

As a matter of fact, when the inflation chamber 4 is filled only with gas, the balloon 1 may have a tendency to rise up into the upper portion of the stomach and impede the penetration of food into the gastric cavity.

The use of ballasting means thus makes it possible to improve the positioning of the balloon 1 inside the stomach and, at the same time, to eliminate a significant source of discomfort for the patient.

In a particularly advantageous way, the solid and dense bodies 8 are joined together by thread portions 9, so as to form a chain, which is advantageously arranged inside the inflation chamber 4 and preferably attached to the valve 10.

The final placement of the intragastric balloon 1 in accordance with the invention will now be described.

Prior to implantation, the intragastric balloon 1 in accordance with the invention is preferably in a folded-over form, or even compressed, so as to facilitate its insertion into and passage through the oral passages of the patient, and in particular the esophagus.

Advantageously, the balloon 1 may be folded over inside of a holding cover designed to facilitate implantation of the balloon. In this case, the balloon is inserted into the gastric cavity along with its cover and then released, and then the cover is pulled out.

Once positioned inside the stomach of the patient, the intragastric balloon 1 must undergo several operations on the part of the medical practitioner in order to render it functional, i.e., to give it a sufficient volume so that it occupies a portion of the space of the gastric cavity reserved for food.

In the case of the balloon shown in FIG. 1, the medical practitioner connects the valve 10 to a fluid source, preferentially air, e.g., by means of a catheter extended by an inflating needle, in order to inflate the inflation chamber 4. The purpose of this operation is to enable the expansion of said inflation chamber 4, which will come to exert stress on the outer casing 2, thereby tending to gradually push it out in a centrifugal direction along arrows F1 to F6.

The inflating operation continues in this way until the outer casing 2 attains its expanded and functional configuration.

When this configuration has been attained, the medical practitioner can remove from the body of the patient all of the material required for inflating, namely the catheter and the inflating needle, the purpose of this operation being to release the balloon.

The system formed by the association of the inflation chamber 4 with the outer casing 2 thus has several characteristics in common with the conventional air chamber-tire pair, particularly in terms of flexibility and elasticity, but also in terms of mechanical strength and protection of the system from external stresses.

The intragastric balloon 1 according to the invention is therefore advantageously designed so as to retain its functional shape and volume over the course of time, even though the outer casing 2 might gradually deteriorate.

Another advantage of the intragastric balloon in accordance with the invention is that it has particularly valuable mechanical strength properties, owing, in particular, to the outer casing 2 forming a mechanical protection means for the balloon.

Another advantage of the intragastric balloon 1 in accordance with the invention is that it has an improved leak tightness in comparison with known balloons.

Another advantage of the intragastric balloon 1 in accordance with the invention is that it is sufficiently sturdy to enable easy handling by the medical practitioner.

### POSSIBILITY OF INDUSTRIAL APPLICATION

The invention finds its industrial application in the design and manufacture of implantable devices for controlling obesity.

1. An expandable intragastric balloon designed to be implanted in the stomach of a patient for the treatment of obesity, comprising:
(a) an outer casing that is sufficiently flexible to pass from a reduced-volume configuration to an expanded configuration, thereby imparting the balloon with its functional shape, and:
(b) means for forming the outer casing, which are structurally integrated into the balloon and primarily separate from the outer casing, said forming means being capable of being actuated, once the balloon has been implanted, on the one hand, in order to exert a sufficient driving pressure on the outer casing to force it to deploy and, on the other hand, to occupy a sufficient volume inside said outer casing to ensure the deployment of the outer casing from its reduced-volume configuration to its expanded configuration.

2. The intragastric balloon of claim 1, wherein the forming means comprises an inflation chamber, which is different from the outer casing and which is arranged inside thereof so as to ensure its formation by the introduction of an inflating fluid into said inflation chamber.

3. The intragastric balloon of claim 2, wherein the inflation chamber and the outer casing are shaped such that, when the inflation chamber occupies its expanded position, the outer casing substantially matches the shape of said inflation chamber.

4. The intragastric balloon of claim 2, wherein the inflation chamber comprises an inner pouch that is sufficiently flexible to pass from a reduced-volume position to an expanded position.

5. The intragastric balloon of claim 4, wherein the inner pouch comprises an elastomer material.

6. The intragastric balloon of claim 4, wherein the inner pouch is defined by a wall including at least one shield that is substantially impervious to gases.

7. The intragastric balloon of claim 6, wherein the shield comprises at least one polymer having a gas barrier effect.

8. The intragastric balloon of claim 7, wherein the shield comprises one or more thermoplastic polymers having a gas barrier effect.

9. The intragastric balloon of claim 8, wherein the shield comprises one or more polymers selected from the group of polymers consisting of ethylene vinyl alcohol (EVOH), poly(vinylidene chloride) (PVDC), polyacrylonitrile (PAN), polyamide (PA), bi-oriented polyamide, poly(ethylene terephthalate) (PET), bi-oriented poly(ethylene terephthalate), and thermoplastic elastomer polyurethane.

10. The intragastric balloon of claim 4, wherein the inner pouch comprises at least one thermoplastic elastomer polyurethane film.

11. The intragastric balloon of the claim 1, wherein the outer casing comprises a bio-compatible material.

12. The intragastric balloon of the claim 1, wherein the outer casing comprises an elastomer material.

13. The intragastric balloon of claim 12, wherein the outer casing is made of silicone.

14. The intragastric balloon of claim 13, wherein the silicone is colored white by treating with barium sulfate.

15. The intragastric balloon of the claim 1, wherein the outer casing is covered with parylene.

16. The intragastric balloon of claim 2, wherein the inflating fluid is a gas.

17. The intragastric balloon of claim 2, wherein the inflation chamber is designed to have a substantially spherical shape in its expanded position.

18. The intragastric balloon of claim 2, wherein the inflation chamber and the outer casing are substantially movable in relation to each other.

19. The intragastric balloon of claim 2, wherein the inflation chamber and the outer casing are substantially concentric.

20. The intragastric balloon of claim 2, wherein the inflation chamber is covered with parylene.

21. The intragastric balloon of claim 3, wherein the inflation chamber comprises an inner pouch that is sufficiently flexible to pass from a reduced-volume position to an expanded position.

22. The intragastric balloon of claim 5, wherein the inner pouch is defined by a wall including at least one shield that is substantially impervious to gases.
